(19) United States
(12) Patent Application Publication
(10) Pub. No.: US 2024/0230688 A1
(43) Pub. Date: Jul. 11, 2024

(54) METHOD FOR DETERMINING PERSONALIZED OMEGA-3 DOSE

(71) Applicant: SOCIETE DES PRODUITS NESTLE S.A., Vevey (CH)

(72) Inventors: SAGAR THAKKAR, La Tour de Peilz (CH); IRMA SILVA ZOLEZZI, Jongny (CH); SURABHI DEVARAJ, Fort Gardens (SG); FANG HUANG, Beijing (CN); JACKLYN RUILIN CHEN, Singapore (SG); CARLOS ANTONIO DE CASTRO, Singapore (SG)

(21) Appl. No.: 18/559,869

(22) PCT Filed: May 9, 2022
(86) PCT No.: PCT/EP2022/062466

§ 371 Date: Nov. 9, 2023

(30) Foreign Application Priority Data
May 10, 2021 (EP) 21173074.2

(51) Int. Cl. G01N 33/92

(52) U.S. Cl. CPC G01N 33/92; G01N 2800/368; G01N 2800/52

(57) ABSTRACT

The present invention relates to a method for determining a personalised omega-3 dose to be taken by a pregnant woman to promote a full-term birth.

### FIELD OF THE INVENTION

The present invention relates to a method for determining a personalised omega-3 dose to be taken by a pregnant woman to promote a full-term birth.

### BACKGROUND TO THE INVENTION

Omega-3 fatty acids are essential fatty acids that must be consumed in the diet. Adequate consumption of omega-3 long-chain polyunsaturated fatty acids (LCPUFA) is vitally important during pregnancy as they are critical building blocks of fetal brain and retina. However, pregnant women in many countries eat little fish and therefore do not consume enough omega-3 LCPUFA (Coletta, J. M., et al., 2010. Reviews in obstetrics and gynecology, 3(4), p. 163).

In particular, it has been found that preterm birth and early preterm birth are reduced in women receiving omega-3 LCPUFA compared with no omega-3. There is also a possibly reduced risk of perinatal death and of neonatal care admission and a reduced risk of LBW babies. (Middleton, P., et al., 2018. Cochrane Database of Systematic Reviews, 11). Omega-3 LCPUFA, particularly docosahexaenoic acid (DHA), supplementation during pregnancy is thus a simple and effective way to reduce preterm, early preterm birth and low birthweight, with low cost and little indication of harm (Middleton, P., et al., 2018. Cochrane Database of Systematic Reviews, 11).

A universal strategy of supplementation has been proposed, however ideally supplementation should be targeted to women who would benefit the most. Existing supplementation recommendations do not target pregnant women who would most benefit from omega-3 LCPUFA supplementation and ignore the overdose risk which may, for example, increase the risk of early preterm birth.

Thus, there is a demand for new personalised omega-3 doses to be taken by pregnant women.

### SUMMARY OF THE INVENTION

The inventors have developed a new method for determining a personalised omega-3 dose to be taken by a pregnant woman. The method uses the woman's omega-3 level, and optionally one or more additional parameters, to determine the woman's personalised omega-3 dose. The personalised omega-3 dose may promote a full-term birth and/or reduce the risk or a preterm/early preterm birth.

In one aspect, the present invention provides a method for determining a personalised omega-3 dose to be taken by a pregnant woman to promote a full-term birth, wherein the method comprises using the woman's omega-3 level to determine the personalised omega-3 dose. Optionally, the method further comprises using one or more additional parameters to determine the personalised omega-3 dose.

In some embodiments, the one or more additional parameters comprise one or more parameter selected from: the woman's current omega-3 consumption, the woman's age, the woman's current weight, the woman's pre-pregnancy weight, the woman's height, the woman's medical history, the woman's lifestyle, the woman's genes, the duration of the woman's current pregnancy, the woman's race or ethnicity, and the woman's integrated socioeconomic status score. In preferred embodiments, the one or more additional parameters comprise one or more parameter selected from: the woman's current omega-3 consumption, the woman's age, the woman's current weight, the woman's pre-pregnancy weight, the woman's height, the woman's medical history, the woman's lifestyle, and the woman's genes. In more preferred embodiments, the one or more additional parameters comprise one or more parameter selected from: the woman's current omega-3 consumption, the woman's age, the woman's current weight, the woman's pre-pregnancy weight, and the woman's height.

Suitably, the woman's medical history comprises one or more parameter selected from: pre-eclampsia or eclampsia in a previous pregnancy, previous pre-term birth, fertility treatment in current pregnancy, woman's compliance, type 2 diabetes, gestational diabetes mellitus in previous pregnancy, woman born preterm, woman's gravidity, woman's parity, any previous pregnancy ended before twenty weeks, previous stillbirth, and previous neonatal death. Preferably, the woman's medical history comprises one or more parameter selected from: pre-eclampsia or eclampsia in a previous pregnancy, previous pre-term birth, fertility treatment in current pregnancy, woman's compliance, type 2 diabetes, and gestational diabetes mellitus in previous pregnancy. Suitably, the woman's lifestyle comprises smoking status and/or alcohol consumption.

In some embodiments:
(i) if the omega-3 level is equal to or below a first threshold, the personalised omega-3 dose is an omega-3 supplementation dose;
(ii) if the omega-3 level is above the first threshold but below a second threshold the personalised omega-3 dose is an omega-3 maintenance dose; and
(iii) if the omega-3 level is equal to or above the second threshold the personalised omega-3 dose is an omega-3 low dose or an omega-3 zero dose.

In some embodiments, the first threshold and the second threshold are determined using the woman's relative risk of early preterm birth (RR EPTB). In some embodiments, the woman's RR EPTB is adjusted for the one or more additional parameters. Suitably, the first threshold is an omega-3 level at and/or below which the woman's RR EPTB is significantly less than one and/or the second threshold is an omega-3 level at and/or above which the woman's RR EPTB is significantly more than one. Suitably, the woman's RR EPTB is significantly less than one if the 95% confidence interval is less than one, and the woman's RR EPTB is significantly more than one if the 95% confidence interval is more than one.

Suitably, the first threshold is an omega-3 level of 4.5% or less, 4.4% or less, 4.3% or less, 4.2% or less, or 4.1% or less. Suitably, the first threshold is an omega-3 level of 3.5% or more, 3.6% or more, 3.7% or more, 3.8% or more, 3.9% or more, 4.0% or more, or 4.1% or more. Suitably, the first threshold is an omega-3 level of from 3.5% to less than 4.5%, from 3.7% to 4.4%, from 3.9% to 4.3%, from 4.0% to 4.2%, or about 4.1%.

Suitably, the second threshold is an omega-3 level of more than 4.5%, 4.6% or more, 4.7% or more, 4.8% or more, or 4.9% or more. Suitably, the second threshold is an omega-3 level of 5.5% or less, 5.4% or less, 5.3% or less, 5.2% or less, 5.1% or less, 5.0% or less, or 4.9% or less. Suitably, the second threshold is an omega-3 level of from more than 4.5% to 5.5%, from 4.6% to 5.3%, from 4.7% to 5.1%, from 4.8% to 5.0%, or about 4.9%.

The omega-3 level may be the total amount of omega-3 fatty acids as a percentage of total fatty acids in the woman's blood. In some embodiments, the omega-3 fatty acids comprise or consist of docosahexaenoic acid (DHA), eicosapentaenoic acid (EPA), docosapentaenoic acid (DPA), and alpha-linolenic acid (ALA). Suitably, the omega-3 level is determined prior to 20 weeks of gestation, preferably wherein the omega-3 level is determined at 12 to 16 weeks of gestation or about 14 weeks of gestation. Suitably, the omega-3 level is determined using dried blood spot analysis, a home-based test kit, or conventional blood analysis, preferably dried spot analysis.

The personalised omega-3 dose may be a daily dose. Suitably, the omega-3 supplementation dose provides from 300 to 1000 mg omega-3 fatty acids per day, preferably wherein the omega-3 supplementation dose provides from 640 to 960 mg DHA per day and/or from 80 to 120 mg EPA per day, more preferably wherein the omega-3 supplementation dose provides about 800 mg DHA and about 100 mg EPA per day. Suitably, the omega-3 maintenance dose provides from 100 to 300 mg omega-3 fatty acids per day, preferably wherein the omega-3 maintenance dose provides from 160 to 240 mg DHA per day and/or from 20 to 30 mg EPA per day, more preferably wherein the omega-3 maintenance dose provides about 200 mg DHA and about 25 mg EPA per day. Suitably, the omega-3 low dose provides less than 100 mg omega-3 fatty acids per day. Suitably, the omega-3 low dose and/or the omega-3 zero dose provides a multivitamin and mineral supplement. In some embodiments, the omega-3 supplementation dose, omega-3 maintenance dose, omega-3 low dose, and/or omega-3 zero dose is adjusted for the one or more additional parameters.

In some embodiments, the method is a computer-implemented method.

In another aspect, the present invention provides a method for determining a personalised omega-3 dose to be taken by a pregnant woman to promote a full-term birth, wherein the method comprises:
(i) determining the woman's omega-3 level; and
(ii) determining the personalised omega-3 dose using the method according to the present invention.

In another aspect, the present invention provides a method for providing a personalised omega-3 dose to be taken by a pregnant woman to promote a full-term birth, wherein the method comprises:
(i) optionally, determining the woman's omega-3 level;
(ii) determining the personalised omega-3 dose using the method according to the present invention; and
(iii) administering the personalised omega-3 dose to the woman.

In another aspect, the present invention provides a method of promoting a full-term birth for a pregnant woman, the method comprising administering a personalised omega-3 dose to the woman, wherein the personalised omega-3 dose is determined using the method according to the present invention.

In another aspect, the present invention provides a computer program comprising instructions which, when the program is executed by a computer, cause the computer to carry out the method according to the present invention.

In another aspect, the present invention provides a computer program comprising instructions which, when the program is executed by a computer, cause the computer to determine a personalised omega-3 dose to be taken by a pregnant woman to promote a full-term birth, given the woman's omega-3 level and optionally one or more additional parameters.

In another aspect, the present invention provides a computer-readable medium comprising instructions which, when executed by a computer, cause the computer to carry out the method according to the present invention.

In another aspect, the present invention provides a computer-readable medium comprising instructions which, when executed by a computer, cause the computer to determine a personalised omega-3 dose to be taken by a pregnant woman to promote a full-term birth, given the woman's omega-3 level and optionally one or more additional parameters.

In another aspect, the present invention provides use of a personalised omega-3 dose, wherein the personalised omega-3 dose is determined using the method according to the present invention.

### DESCRIPTION OF DRAWINGS

FIG. 1—exemplary chart showing relative risk of early preterm birth in relation to omega-3 level

Adapted from Simmonds, L. A., et al., 2020. BJOG: An International Journal of Obstetrics & Gynaecology, 127(8), pp. 975-981. The zone on the left shows that an omega-3 supplementation dose reduces the woman's risk of early preterm birth in women with a total omega-3 level of ≤4.1%. The zone on the right shows that an omega-3 supplementation dose increases the woman's risk of early preterm birth in women with a total omega-3 level of >4.9%.

### DETAILED DESCRIPTION

Various preferred features and embodiments of the present invention will now be described by way of non-limiting examples. The skilled person will understand that they can combine all features of the invention disclosed herein without departing from the scope of the invention as disclosed.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

Numeric ranges are inclusive of the numbers defining the range.

The publications discussed herein are provided solely for their disclosure prior to the filing date of the present application. Nothing herein is to be construed as an admission that such publications constitute prior art to the claims appended hereto.

### Method for Determining a Personalised Omega-3 Dose

In one aspect, the present invention provides a method for determining a personalised omega-3 dose to be taken by a pregnant woman.

The personalised omega-3 dose may be determined for any woman at any period before or during pregnancy. The personalised omega-3 dose may be determined one or more times before or during pregnancy. Suitably, the personalised omega-3 dose is determined one or more times during pregnancy. Suitably, the personalised omega-3 dose is determined prior to 20 weeks of gestation, preferably wherein the personalised omega-3 dose is determined at 12 to 16 weeks of gestation, more preferably wherein the personalised omega-3 dose is determined at about 14 weeks of gestation.

The method of the present invention comprises using the woman's omega-3 level, and optionally one or more additional parameters, to determine the personalised omega-3 dose.

### Omega-3 Level

As used herein, the term “omega-3 level” refers to the amount of omega-3 fatty acids in the woman's blood, e.g. the woman's capillary whole blood.

Omega-3 fatty acids (also known as omega-3 oils, ω-3 fatty acids or n-3 fatty acids) are polyunsaturated fatty acids (PUFAs) characterised by the presence of a double bond, three atoms away from the terminal methyl group in their chemical structure. The most common omega-3 fatty acids found in nature include hexadecatrienoic acid (HTA, 16:3 (n-3)), α-Linolenic acid (ALA, 18:3 (n-3)), stearidonic acid (SDA, 18:4 (n-3)), eicosatrienoic acid (ETE, 20:3 (n-3)), eicosatetraenoic acid (ETA, 20:4 (n-3)), eicosapentaenoic acid (EPA, 20:5 (n-3)), heneicosapentaenoic acid (HPA, 21:5 (n-3)), docosapentaenoic acid (DPA, 22:5 (n-3)), docosahexaenoic acid (DHA, 22:6 (n-3)), tetracosapentaenoic acid (24:5 (n-3)), and tetracosahexaenoic acid (24:6 (n-3)).

Suitably, the omega-3 fatty acids are omega-3 long-chain polyunsaturated fatty acids (LCPUFA). LCPUFA may refer to polyunsaturated fatty acids with 18 carbons or more.

Suitably, the omega-3 fatty acids comprise or consist of docosahexaenoic acid (DHA), eicosapentaenoic acid (EPA), docosapentaenoic acid (DPA), and alpha-linolenic acid (ALA). In some embodiments, the omega-3 level is the total amount of DHA, EPA, DPA and ALA in the woman's blood, e.g. the woman's capillary whole blood.

The omega-3 level may be given in any suitable measurement unit. Suitably, the omega-3 level is the total amount of omega-3 fatty acids as a percentage of total fatty acids in the woman's blood, e.g. the woman's capillary whole blood. In some embodiments, the omega-3 level is the total amount of DHA, EPA, DPA and ALA as a percentage of total fatty acids in the woman's blood, e.g. the woman's capillary whole blood.

The omega-3 level may be determined at any period before or during pregnancy. The omega-3 level may be determined one or more times before or during pregnancy. Suitably, the omega-3 level is determined one or more times during pregnancy. Suitably, the omega-3 level is determined prior to 20 weeks of gestation, preferably wherein the omega-3 level is determined at 12 to 16 weeks of gestation, more preferably wherein the omega-3 level is determined at about 14 weeks of gestation.

The method of the present invention may comprise determining the woman's omega-3 level. The omega-3 level may be determined by any suitable method known in the art (see e.g. Klingler, M. and Koletzko, B., 2012. British journal of nutrition, 107(S2), pp. S53-S63). Suitably, the omega-3 level is determined by dried spot analysis, a home-based test kit, and/or conventional blood analysis. In some embodiments, the omega-3 level is determined by dried spot analysis. A method of determining omega-3 level by dried spot analysis is described in Marangoni, F., et al., 2004. Analytical biochemistry, 326(2), pp. 267-272 and Harris, W. S. and Polreis, J., 2016. Ann Clin Lab Res, 4(4). In some embodiments, the omega-3 level is determined by a home-based test kit. Suitable home-based test kits are commercially available. The blood (e.g. capillary whole blood) may be obtained or obtainable by any suitable method known in the art. Suitably, the blood (e.g. capillary whole blood) is obtained or obtainable by finger prick or venipuncture.

### Additional Parameters

The method of the present invention may further comprise using one or more additional parameters to determine the personalised omega-3 dose.

In some embodiments, the method of the present invention further comprises using one or more additional parameters (e.g. 1, 2, 3, or 4 parameters) to determine the personalised omega-3 dose. In some embodiments, the method of the present invention further comprises using two or more additional parameters (e.g. 2, 3, or 4 parameters) to determine the personalised omega-3 dose. In some embodiments, the method of the present invention further comprises using three or more additional parameters to determine the personalised omega-3 dose. In some embodiments, the method of the present invention further comprises using four or more additional parameters to determine the personalised omega-3 dose.

Exemplary additional parameters include: the woman's current omega-3 consumption, the woman's age, the woman's current weight, the woman's pre-pregnancy weight, the woman's height, the woman's medical history, the woman's lifestyle, the woman's genes, the duration of the woman's current pregnancy, the woman's race or ethnicity, and the woman's integrated socioeconomic status score.

The additional parameters may be provided at any period before or during pregnancy. The additional parameters may be provided one or more times before or during pregnancy. Suitably, the additional parameters are provided one or more times during pregnancy. Suitably, the additional parameters are provided prior to 20 weeks of gestation, preferably wherein the additional parameters are provided at 12 to 16 weeks of gestation, more preferably wherein the additional parameters are provided at about 14 weeks of gestation.

In some embodiments, the method comprises using the woman's omega-3 level and one or more additional parameters selected from: the woman's current omega-3 consumption, the woman's age, the woman's current weight, the woman's pre-pregnancy weight, the woman's height, the woman's medical history, the woman's lifestyle, the woman's genes, the duration of the woman's current pregnancy, the woman's race or ethnicity, and the woman's integrated socioeconomic status score, to determine the personalised omega-3 dose.

In some embodiments, the method comprises using the woman's omega-3 level and one or more additional parameters selected from: the woman's current omega-3 consumption, the woman's age, the woman's current weight, the woman's pre-pregnancy weight, the woman's height, the woman's medical history, the woman's lifestyle, and the woman's genes, to determine the personalised omega-3 dose.

In some embodiments, the method comprises using the woman's omega-3 level and one or more additional parameters selected from: the woman's current omega-3 consumption, the woman's age, the woman's current weight, the woman's pre-pregnancy weight, and the woman's height, to determine the personalised omega-3 dose.

In some embodiments, the method comprises using the woman's omega-3 level and the woman's current omega-3 consumption to determine the personalised omega-3 dose. In some embodiments, the method comprises using the woman's omega-3 level, the woman's current omega-3 consumption, and one or more additional parameters to determine the personalised omega-3 dose. In some embodiments, the method comprises using the woman's omega-3 level, the woman's current omega-3 consumption, and the woman's age, to determine the personalised omega-3 dose. In some embodiments, the method comprises using the woman's omega-3 level, the woman's current omega-3 consumption, and the woman's current weight, to determine the personalised omega-3 dose. In some embodiments, the method comprises using the woman's omega-3 level, the woman's current omega-3 consumption, and the woman's pre-pregnancy weight, to determine the personalised omega-3 dose. In some embodiments, the method comprises using the woman's omega-3 level, the woman's current omega-3 consumption, and the woman's height, to determine the personalised omega-3 dose. In some embodiments, the method comprises using the woman's omega-3 level, the woman's current omega-3 consumption, and the woman's medical history, to determine the personalised omega-3 dose. In some embodiments, the method comprises using the woman's omega-3 level, the woman's current omega-3 consumption, and the woman's lifestyle, to determine the personalised omega-3 dose. In some embodiments, the method comprises using the woman's omega-3 level, the woman's current omega-3 consumption, and the woman's genes, to determine the personalised omega-3 dose. In some embodiments, the method comprises using the woman's omega-3 level, the woman's current omega-3 consumption, and the duration of the woman's current pregnancy, to determine the personalised omega-3 dose. In some embodiments, the method comprises using the woman's omega-3 level, the woman's current omega-3 consumption, and the woman's race or ethnicity, to determine the personalised omega-3 dose. In some embodiments, the method comprises using the woman's omega-3 level, the woman's current omega-3 consumption, and the woman's integrated socioeconomic status score, to determine the personalised omega-3 dose.

In some embodiments, the method comprises using the woman's omega-3 level and the woman's age to determine the personalised omega-3 dose. In some embodiments, the method comprises using the woman's omega-3 level, the woman's age, and one or more additional parameters to determine the personalised omega-3 dose. In some embodiments, the method comprises using the woman's omega-3 level, the woman's age, and the woman's current omega-3 consumption, to determine the personalised omega-3 dose. In some embodiments, the method comprises using the woman's omega-3 level, the woman's age, and the woman's current weight, to determine the personalised omega-3 dose. In some embodiments, the method comprises using the woman's omega-3 level, the woman's age, and the woman's pre-pregnancy weight, to determine the personalised omega-3 dose. In some embodiments, the method comprises using the woman's omega-3 level, the woman's age, and the woman's height, to determine the personalised omega-3 dose. In some embodiments, the method comprises using the woman's omega-3 level, the woman's age, and the woman's medical history, to determine the personalised omega-3 dose. In some embodiments, the method comprises using the woman's omega-3 level, the woman's age, and the woman's lifestyle, to determine the personalised omega-3 dose. In some embodiments, the method comprises using the woman's omega-3 level, the woman's age, and the woman's genes, to determine the personalised omega-3 dose. In some embodiments, the method comprises using the woman's omega-3 level, the woman's age, and the duration of the woman's current pregnancy, to determine the personalised omega-3 dose. In some embodiments, the method comprises using the woman's omega-3 level, the woman's age, and the woman's race or ethnicity, to determine the personalised omega-3 dose. In some embodiments, the method comprises using the woman's omega-3 level, the woman's age, and the woman's integrated socioeconomic status score, to determine the personalised omega-3 dose.

In some embodiments, the method comprises using the woman's omega-3 level and the woman's current weight to determine the personalised omega-3 dose. In some embodiments, the method comprises using the woman's omega-3 level, the woman's current weight, and one or more additional parameters to determine the personalised omega-3 dose. In some embodiments, the method comprises using the woman's omega-3 level, the woman's current weight, and the woman's current omega-3 consumption, to determine the personalised omega-3 dose. In some embodiments, the method comprises using the woman's omega-3 level, the woman's current weight, and the woman's age, to determine the personalised omega-3 dose. In some embodiments, the method comprises using the woman's omega-3 level, the woman's current weight, and the woman's pre-pregnancy weight, to determine the personalised omega-3 dose. In some embodiments, the method comprises using the woman's omega-3 level, the woman's current weight, and the woman's height, to determine the personalised omega-3 dose. In some embodiments, the method comprises using the woman's omega-3 level, the woman's current weight, and the woman's medical history, to determine the personalised omega-3 dose. In some embodiments, the method comprises using the woman's omega-3 level, the woman's current weight, and the woman's lifestyle, to determine the personalised omega-3 dose. In some embodiments, the method comprises using the woman's omega-3 level, the woman's current weight, and the woman's genes, to determine the personalised omega-3 dose. In some embodiments, the method comprises using the woman's omega-3 level, the woman's current weight, and the duration of the woman's current pregnancy, to determine the personalised omega-3 dose. In some embodiments, the method comprises using the woman's omega-3 level, the woman's current weight, and the woman's race or ethnicity, to determine the personalised omega-3 dose. In some embodiments, the method comprises using the woman's omega-3 level, the woman's current weight, and the woman's integrated socioeconomic status score, to determine the personalised omega-3 dose.

In some embodiments, the method comprises using the woman's omega-3 level and the woman's pre-pregnancy weight to determine the personalised omega-3 dose. In some embodiments, the method comprises using the woman's omega-3 level, the woman's pre-pregnancy weight, and one or more additional parameters to determine the personalised omega-3 dose. In some embodiments, the method comprises using the woman's omega-3 level, the woman's pre-pregnancy weight, and the woman's current omega-3 consumption, to determine the personalised omega-3 dose. In some embodiments, the method comprises using the woman's omega-3 level, the woman's pre-pregnancy weight, and the woman's age, to determine the personalised omega-3 dose. In some embodiments, the method comprises using the woman's omega-3 level, the woman's pre-pregnancy weight, and the woman's current weight, to determine the personalised omega-3 dose. In some embodiments, the method comprises using the woman's omega-3 level, the woman's pre-pregnancy weight, and the woman's height, to determine the personalised omega-3 dose. In some embodiments, the method comprises using the woman's omega-3 level, the woman's pre-pregnancy weight, and the woman's medical history, to determine the personalised omega-3 dose. In some embodiments, the method comprises using the woman's omega-3 level, the woman's pre-pregnancy weight, and the woman's lifestyle, to determine the personalised omega-3 dose. In some embodiments, the method comprises using the woman's omega-3 level, the woman's pre-pregnancy weight, and the woman's genes, to determine the personalised omega-3 dose. In some embodiments, the method comprises using the woman's omega-3 level, the woman's pre-pregnancy weight, and the duration of the woman's current pregnancy, to determine the personalised omega-3 dose. In some embodiments, the method comprises using the woman's omega-3 level, the woman's pre-pregnancy weight, and the woman's race or ethnicity, to determine the personalised omega-3 dose. In some embodiments, the method comprises using the woman's omega-3 level, the woman's pre-pregnancy weight, and the woman's integrated socioeconomic status score, to determine the personalised omega-3 dose.

In some embodiments, the method comprises using the woman's omega-3 level and the woman's height to determine the personalised omega-3 dose. In some embodiments, the method comprises using the woman's omega-3 level, the woman's height, and one or more additional parameters to determine the personalised omega-3 dose. In some embodiments, the method comprises using the woman's omega-3 level, the woman's height, and the woman's current omega-3 consumption, to determine the personalised omega-3 dose. In some embodiments, the method comprises using the woman's omega-3 level, the woman's height, and the woman's age, to determine the personalised omega-3 dose. In some embodiments, the method comprises using the woman's omega-3 level, the woman's height, and the woman's current weight, to determine the personalised omega-3 dose. In some embodiments, the method comprises using the woman's omega-3 level, the woman's height, and the woman's pre-pregnancy weight, to determine the personalised omega-3 dose. In some embodiments, the method comprises using the woman's omega-3 level, the woman's height, and the woman's medical history, to determine the personalised omega-3 dose. In some embodiments, the method comprises using the woman's omega-3 level, the woman's height, and the woman's lifestyle, to determine the personalised omega-3 dose. In some embodiments, the method comprises using the woman's omega-3 level, the woman's height, and the woman's genes, to determine the personalised omega-3 dose. In some embodiments, the method comprises using the woman's omega-3 level, the woman's height, and the duration of the woman's current pregnancy, to determine the personalised omega-3 dose. In some embodiments, the method comprises using the woman's omega-3 level, the woman's height, and the woman's race or ethnicity, to determine the personalised omega-3 dose. In some embodiments, the method comprises using the woman's omega-3 level, the woman's height, and the woman's integrated socioeconomic status score, to determine the personalised omega-3 dose.

In some embodiments, the method comprises using the woman's omega-3 level and the woman's medical history to determine the personalised omega-3 dose. In some embodiments, the method comprises using the woman's omega-3 level, the woman's medical history, and one or more additional parameters to determine the personalised omega-3 dose. In some embodiments, the method comprises using the woman's omega-3 level, the woman's medical history, and the woman's current omega-3 consumption, to determine the personalised omega-3 dose. In some embodiments, the method comprises using the woman's omega-3 level, the woman's medical history, and the woman's age, to determine the personalised omega-3 dose. In some embodiments, the method comprises using the woman's omega-3 level, the woman's medical history, and the woman's current weight, to determine the personalised omega-3 dose. In some embodiments, the method comprises using the woman's omega-3 level, the woman's medical history, and the woman's pre-pregnancy weight, to determine the personalised omega-3 dose. In some embodiments, the method comprises using the woman's omega-3 level, the woman's medical history, and the woman's height, to determine the personalised omega-3 dose. In some embodiments, the method comprises using the woman's omega-3 level, the woman's medical history, and the woman's lifestyle, to determine the personalised omega-3 dose. In some embodiments, the method comprises using the woman's omega-3 level, the woman's medical history, and the woman's genes, to determine the personalised omega-3 dose. In some embodiments, the method comprises using the woman's omega-3 level, the woman's medical history, and the duration of the woman's current pregnancy, to determine the personalised omega-3 dose. In some embodiments, the method comprises using the woman's omega-3 level, the woman's medical history, and the woman's race or ethnicity, to determine the personalised omega-3 dose. In some embodiments, the method comprises using the woman's omega-3 level, the woman's medical history, and the woman's integrated socioeconomic status score, to determine the personalised omega-3 dose.

In some embodiments, the method comprises using the woman's omega-3 level and the woman's lifestyle to determine the personalised omega-3 dose. In some embodiments, the method comprises using the woman's omega-3 level, the woman's lifestyle, and one or more additional parameters to determine the personalised omega-3 dose. In some embodiments, the method comprises using the woman's omega-3 level, the woman's lifestyle, and the woman's current omega-3 consumption, to determine the personalised omega-3 dose. In some embodiments, the method comprises using the woman's omega-3 level, the woman's lifestyle, and the woman's age, to determine the personalised omega-3 dose. In some embodiments, the method comprises using the woman's omega-3 level, the woman's lifestyle, and the woman's current weight, to determine the personalised omega-3 dose. In some embodiments, the method comprises using the woman's omega-3 level, the woman's lifestyle, and the woman's pre-pregnancy weight, to determine the personalised omega-3 dose. In some embodiments, the method comprises using the woman's omega-3 level, the woman's lifestyle, and the woman's height, to determine the personalised omega-3 dose. In some embodiments, the method comprises using the woman's omega-3 level, the woman's lifestyle, and the woman's medical history, to determine the personalised omega-3 dose. In some embodiments, the method comprises using the woman's omega-3 level, the woman's lifestyle, and the woman's genes, to determine the personalised omega-3 dose. In some embodiments, the method comprises using the woman's omega-3 level, the woman's lifestyle, and the duration of the woman's current pregnancy, to determine the personalised omega-3 dose. In some embodiments, the method comprises using the woman's omega-3 level, the woman's lifestyle, and the woman's race or ethnicity, to determine the personalised omega-3 dose. In some embodiments, the method comprises using the woman's omega-3 level, the woman's lifestyle, and the woman's integrated socioeconomic status score, to determine the personalised omega-3 dose.

In some embodiments, the method comprises using the woman's omega-3 level and the woman's genes to determine the personalised omega-3 dose. In some embodiments, the method comprises using the woman's omega-3 level, the woman's genes, and one or more additional parameters to determine the personalised omega-3 dose. In some embodiments, the method comprises using the woman's omega-3 level, the woman's genes, and the woman's current omega-3 consumption, to determine the personalised omega-3 dose. In some embodiments, the method comprises using the woman's omega-3 level, the woman's genes, and the woman's age, to determine the personalised omega-3 dose. In some embodiments, the method comprises using the woman's omega-3 level, the woman's genes, and the woman's current weight, to determine the personalised omega-3 dose. In some embodiments, the method comprises using the woman's omega-3 level, the woman's genes, and the woman's pre-pregnancy weight, to determine the personalised omega-3 dose. In some embodiments, the method comprises using the woman's omega-3 level, the woman's genes, and the woman's height, to determine the personalised omega-3 dose. In some embodiments, the method comprises using the woman's omega-3 level, the woman's genes, and the woman's medical history, to determine the personalised omega-3 dose. In some embodiments, the method comprises using the woman's omega-3 level, the woman's genes, and the woman's lifestyle, to determine the personalised omega-3 dose. In some embodiments, the method comprises using the woman's omega-3 level, the woman's genes, and the duration of the woman's current pregnancy, to determine the personalised omega-3 dose. In some embodiments, the method comprises using the woman's omega-3 level, the woman's genes, and the woman's race or ethnicity, to determine the personalised omega-3 dose. In some embodiments, the method comprises using the woman's omega-3 level, the woman's genes, and the woman's integrated socioeconomic status score, to determine the personalised omega-3 dose.

In some embodiments, the method comprises using the woman's omega-3 level and the duration of the woman's current pregnancy to determine the personalised omega-3 dose. In some embodiments, the method comprises using the woman's omega-3 level, the duration of the woman's current pregnancy, and one or more additional parameters to determine the personalised omega-3 dose. In some embodiments, the method comprises using the woman's omega-3 level, the duration of the woman's current pregnancy, and the woman's current omega-3 consumption, to determine the personalised omega-3 dose. In some embodiments, the method comprises using the woman's omega-3 level, the duration of the woman's current pregnancy, and the woman's age, to determine the personalised omega-3 dose. In some embodiments, the method comprises using the woman's omega-3 level, the duration of the woman's current pregnancy, and the woman's current weight, to determine the personalised omega-3 dose. In some embodiments, the method comprises using the woman's omega-3 level, the duration of the woman's current pregnancy, and the woman's pre-pregnancy weight, to determine the personalised omega-3 dose. In some embodiments, the method comprises using the woman's omega-3 level, the duration of the woman's current pregnancy, and the woman's height, to determine the personalised omega-3 dose. In some embodiments, the method comprises using the woman's omega-3 level, the duration of the woman's current pregnancy, and the woman's medical history, to determine the personalised omega-3 dose. In some embodiments, the method comprises using the woman's omega-3 level, the duration of the woman's current pregnancy, and the woman's lifestyle, to determine the personalised omega-3 dose. In some embodiments, the method comprises using the woman's omega-3 level, the duration of the woman's current pregnancy, and the woman's genes, to determine the personalised omega-3 dose. In some embodiments, the method comprises using the woman's omega-3 level, the duration of the woman's current pregnancy, and the woman's race or ethnicity, to determine the personalised omega-3 dose. In some embodiments, the method comprises using the woman's omega-3 level, the duration of the woman's current pregnancy, and the woman's integrated socioeconomic status score, to determine the personalised omega-3 dose.

In some embodiments, the method comprises using the woman's omega-3 level and the woman's race or ethnicity to determine the personalised omega-3 dose. In some embodiments, the method comprises using the woman's omega-3 level, the woman's race or ethnicity, and one or more additional parameters to determine the personalised omega-3 dose. In some embodiments, the method comprises using the woman's omega-3 level, the woman's race or ethnicity, and the woman's current omega-3 consumption, to determine the personalised omega-3 dose. In some embodiments, the method comprises using the woman's omega-3 level, the woman's race or ethnicity, and the woman's age, to determine the personalised omega-3 dose. In some embodiments, the method comprises using the woman's omega-3 level, the woman's race or ethnicity, and the woman's current weight, to determine the personalised omega-3 dose. In some embodiments, the method comprises using the woman's omega-3 level, the woman's race or ethnicity, and the woman's pre-pregnancy weight, to determine the personalised omega-3 dose. In some embodiments, the method comprises using the woman's omega-3 level, the woman's race or ethnicity, and the woman's height, to determine the personalised omega-3 dose. In some embodiments, the method comprises using the woman's omega-3 level, the woman's race or ethnicity, and the woman's medical history, to determine the personalised omega-3 dose. In some embodiments, the method comprises using the woman's omega-3 level, the woman's race or ethnicity, and the woman's lifestyle, to determine the personalised omega-3 dose. In some embodiments, the method comprises using the woman's omega-3 level, the woman's race or ethnicity, and the woman's genes, to determine the personalised omega-3 dose. In some embodiments, the method comprises using the woman's omega-3 level, the woman's race or ethnicity, and the duration of the woman's current pregnancy, to determine the personalised omega-3 dose. In some embodiments, the method comprises using the woman's omega-3 level, the woman's race or ethnicity, and the woman's integrated socioeconomic status score, to determine the personalised omega-3 dose.

In some embodiments, the method comprises using the woman's omega-3 level and the woman's integrated socioeconomic status score to determine the personalised omega-3 dose. In some embodiments, the method comprises using the woman's omega-3 level, the woman's integrated socioeconomic status score, and one or more additional parameters to determine the personalised omega-3 dose. In some embodiments, the method comprises using the woman's omega-3 level, the woman's integrated socioeconomic status score, and the woman's current omega-3 consumption, to determine the personalised omega-3 dose. In some embodiments, the method comprises using the woman's omega-3 level, the woman's integrated socioeconomic status score, and the woman's age, to determine the personalised omega-3 dose. In some embodiments, the method comprises using the woman's omega-3 level, the woman's integrated socioeconomic status score, and the woman's current weight, to determine the personalised omega-3 dose. In some embodiments, the method comprises using the woman's omega-3 level, the woman's integrated socioeconomic status score, and the woman's pre-pregnancy weight, to determine the personalised omega-3 dose. In some embodiments, the method comprises using the woman's omega-3 level, the woman's integrated socioeconomic status score, and the woman's height, to determine the personalised omega-3 dose. In some embodiments, the method comprises using the woman's omega-3 level, the woman's integrated socioeconomic status score, and the woman's medical history, to determine the personalised omega-3 dose. In some embodiments, the method comprises using the woman's omega-3 level, the woman's integrated socioeconomic status score, and the woman's lifestyle, to determine the personalised omega-3 dose. In some embodiments, the method comprises using the woman's omega-3 level, the woman's integrated socioeconomic status score, and the woman's genes, to determine the personalised omega-3 dose. In some embodiments, the method comprises using the woman's omega-3 level, the woman's integrated socioeconomic status score, and the duration of the woman's current pregnancy, to determine the personalised omega-3 dose. In some embodiments, the method comprises using the woman's omega-3 level, the woman's integrated socioeconomic status score, and the woman's race or ethnicity, to determine the personalised omega-3 dose.

The woman's current omega-3 consumption may include the woman's current omega-3 supplement consumption and/or the woman's current omega-3 dietary consumption. The woman's current omega-3 consumption may comprise or consist of DHA and/or EPA consumption, e.g. the woman's current omega-3 consumption may be the woman's current DHA and/or EPA consumption. Suitably, the woman's current omega-3 consumption is the woman's current DHA and/or EPA supplement consumption. The woman's current omega-3 consumption may be given in any measurement unit, suitably mg per day.

The woman's age may be given in any measurement unit, suitably years. The woman's current weight and pre-pregnancy weight may be given in any suitable measurement unit, suitably kilograms. The woman's height may be given in any suitable measurement unit, suitably cm.

The woman's medical history may include any past illnesses and treatments. For example, the woman's medical history may include one or more of: pre-eclampsia or eclampsia in a previous pregnancy, previous pre-term birth, fertility treatment in current pregnancy, woman's compliance, type 2 diabetes, gestational diabetes mellitus in previous pregnancy, woman born preterm, woman's gravidity, woman's parity, any previous pregnancy ended before twenty weeks, previous stillbirth, and previous neonatal death.

In some embodiments, the woman's medical history comprises one or more parameter selected from: pre-eclampsia or eclampsia in a previous pregnancy, previous pre-term birth, fertility treatment in current pregnancy, woman's compliance, type 2 diabetes, gestational diabetes mellitus in previous pregnancy, woman born preterm, woman's gravidity, woman's parity, any previous pregnancy ended before twenty weeks, previous stillbirth, and previous neonatal death.

In some embodiments, the woman's medical history comprises one or more parameter selected from: pre-eclampsia or eclampsia in a previous pregnancy, previous pre-term birth, fertility treatment in current pregnancy, woman's compliance, type 2 diabetes, and gestational diabetes mellitus in previous pregnancy.

The woman's lifestyle may include any set of habits and customs influenced by life-long process of socialization, including social use of substances such as alcohol and tobacco, and exercise. In some embodiments, the woman's lifestyle comprises smoking status and/or alcohol consumption.

The woman's genes may include any relevant genes. For example, genes involved in metabolism of omega-3 fatty acids (see e.g. Simopoulos, A. P., 2010. Experimental biology and medicine, 235(7), pp. 785-795). Exemplary genes include FADS1 and FADS2.

The duration of the woman's current pregnancy may be given in any measurement unit, suitably weeks.

### Personalised Omega-3 Dose

The woman's omega-3 level, and optionally the one or more additional parameters, may be used to determine the woman's personalised omega-3 dose.

The personalised omega-3 dose may be for any time interval. For example, the personalised omega-3 dose may be a daily dose, a monthly dose, or a yearly dose. In some embodiments, the personalised omega-3 dose is a daily dose.

The personalised omega-3 dose may be given in any measurement unit. For example, the personalised omega-3 dose may be given in mg/day.

Suitably, the omega-3 fatty acids are omega-3 LCPUFA. Suitably, the omega-3 fatty acids comprise or consist of docosahexaenoic acid (DHA) and eicosapentaenoic acid (EPA).

A DHA+EPA dose of up to 3,000 mg per day is generally considered safe. The personalised omega-3 dose may be between 0 mg/day and 3,000 mg/day of DHA+EPA. Suitably, the personalised omega-3 dose may be between 0 mg/day and 1,000 mg/day of DHA+EPA. For example, the personalised omega-3 dose may provide 87.5 to 875 mg DHA and/or 12.5 to 125 mg EPA.

The omega-3 dose may be adjusted for the one or more additional parameters. Suitably, the omega-3 dose may be adjusted for the woman's current omega-3 consumption, the woman's age, the woman's current weight, the woman's pre-pregnancy weight, the woman's height, the woman's medical history, the woman's lifestyle, the woman's genes, the duration of the woman's current pregnancy, the woman's race or ethnicity, and the woman's integrated socioeconomic status score.

For example, the omega-3 dose may be adjusted for women with pre-pregnancy obesity because it is known that maternal pre-pregnancy obesity attenuates response to omega-3 fatty acids supplementation during pregnancy (Monthé-Drèze, C., et al., 2018. Nutrients, 10(12), p. 190). For example, the omega-3 dose may be adjusted for women with genetic variants of the FADS1 FADS2 gene cluster because it is known that genetic variants of the FADS1 FADS2 gene cluster are associated with altered (n-3) essential fatty acids in plasma in women during pregnancy (Xie, L. and Innis, S. M., 2008. The Journal of nutrition, 138(11), pp. 2222-2228).

Any suitable method may be used to adjust the omega-3 dose for the one or more additional parameters. For example, multivariate regression models may be applied to estimate the effect on omega-3 intervention of modifying the one or more additional parameters and the omega-3 dose may be adjusted accordingly. Suitably, the omega-3 dose is adjusted for the one or more additional parameters using a multivariate regression model.

In some embodiments, the omega-3 dose is adjusted for the woman's current omega-3 consumption. In some embodiments, the omega-3 dose is adjusted for the woman's age. In some embodiments, the omega-3 dose is adjusted for the woman's current weight. In some embodiments, the omega-3 dose is adjusted for the woman's pre-pregnancy weight. In some embodiments, the omega-3 dose is adjusted for the woman's height. In some embodiments, the omega-3 dose is adjusted for the woman's medical history. In some embodiments, the omega-3 dose is adjusted for the woman's lifestyle. In some embodiments, the omega-3 dose is adjusted for the woman's genes. In some embodiments, the omega-3 dose is adjusted for the duration of the woman's current pregnancy. In some embodiments, the omega-3 dose is adjusted for the woman's race or ethnicity. In some embodiments, the omega-3 dose is adjusted for the woman's integrated socioeconomic status score.

In some embodiments, the omega-3 dose is adjusted for the woman's current omega-3 consumption and one or more additional parameters. In some embodiments, the omega-3 dose is adjusted for the woman's current omega-3 consumption and the woman's age. In some embodiments, the omega-3 dose is adjusted for the woman's current omega-3 consumption and the woman's current weight. In some embodiments, the omega-3 dose is adjusted for the woman's current omega-3 consumption and the woman's pre-pregnancy weight. In some embodiments, the omega-3 dose is adjusted for the woman's current omega-3 consumption and the woman's height. In some embodiments, the omega-3 dose is adjusted for the woman's current omega-3 consumption and the woman's medical history. In some embodiments, the omega-3 dose is adjusted for the woman's current omega-3 consumption and the woman's lifestyle. In some embodiments, the omega-3 dose is adjusted for the woman's current omega-3 consumption and the woman's genes. In some embodiments, the omega-3 dose is adjusted for the woman's current omega-3 consumption and the duration of the woman's current pregnancy. In some embodiments, the omega-3 dose is adjusted for the woman's current omega-3 consumption and the woman's race or ethnicity. In some embodiments, the omega-3 dose is adjusted for the woman's current omega-3 consumption and the woman's integrated socioeconomic status score.

In some embodiments, the omega-3 dose is adjusted for the woman's age and one or more additional parameters. In some embodiments, the omega-3 dose is adjusted for the woman's age and the woman's current omega-3 consumption. In some embodiments, the omega-3 dose is adjusted for the woman's age and the woman's current weight. In some embodiments, the omega-3 dose is adjusted for the woman's age and the woman's pre-pregnancy weight. In some embodiments, the omega-3 dose is adjusted for the woman's age and the woman's height. In some embodiments, the omega-3 dose is adjusted for the woman's age and the woman's medical history. In some embodiments, the omega-3 dose is adjusted for the woman's age and the woman's lifestyle. In some embodiments, the omega-3 dose is adjusted for the woman's age and the woman's genes. In some embodiments, the omega-3 dose is adjusted for the woman's age and the duration of the woman's current pregnancy. In some embodiments, the omega-3 dose is adjusted for the woman's age and the woman's race or ethnicity. In some embodiments, the omega-3 dose is adjusted for the woman's age and the woman's integrated socioeconomic status score.

In some embodiments, the omega-3 dose is adjusted for the woman's current weight and one or more additional parameters. In some embodiments, the omega-3 dose is adjusted for the woman's current weight and the woman's current omega-3 consumption. In some embodiments, the omega-3 dose is adjusted for the woman's current weight and the woman's age. In some embodiments, the omega-3 dose is adjusted for the woman's current weight and the woman's pre-pregnancy weight. In some embodiments, the omega-3 dose is adjusted for the woman's current weight and the woman's height. In some embodiments, the omega-3 dose is adjusted for the woman's current weight and the woman's medical history. In some embodiments, the omega-3 dose is adjusted for the woman's current weight and the woman's lifestyle. In some embodiments, the omega-3 dose is adjusted for the woman's current weight and the woman's genes. In some embodiments, the omega-3 dose is adjusted for the woman's current weight and the duration of the woman's current pregnancy. In some embodiments, the omega-3 dose is adjusted for the woman's current weight and the woman's race or ethnicity. In some embodiments, the omega-3 dose is adjusted for the woman's current weight and the woman's integrated socioeconomic status score.

In some embodiments, the omega-3 dose is adjusted for the woman's pre-pregnancy weight and one or more additional parameters. In some embodiments, the omega-3 dose is adjusted for the woman's pre-pregnancy weight and the woman's current omega-3 consumption. In some embodiments, the omega-3 dose is adjusted for the woman's pre-pregnancy weight and the woman's age. In some embodiments, the omega-3 dose is adjusted for the woman's pre-pregnancy weight and the woman's current weight. In some embodiments, the omega-3 dose is adjusted for the woman's pre-pregnancy weight and the woman's height. In some embodiments, the omega-3 dose is adjusted for the woman's pre-pregnancy weight and the woman's medical history. In some embodiments, the omega-3 dose is adjusted for the woman's pre-pregnancy weight and the woman's lifestyle. In some embodiments, the omega-3 dose is adjusted for the woman's pre-pregnancy weight and the woman's genes. In some embodiments, the omega-3 dose is adjusted for the woman's pre-pregnancy weight and the duration of the woman's current pregnancy. In some embodiments, the omega-3 dose is adjusted for the woman's pre-pregnancy weight and the woman's race or ethnicity. In some embodiments, the omega-3 dose is adjusted for the woman's pre-pregnancy weight and the woman's integrated socioeconomic status score.

In some embodiments, the omega-3 dose is adjusted for the woman's height and one or more additional parameters. In some embodiments, the omega-3 dose is adjusted for the woman's height and the woman's current omega-3 consumption. In some embodiments, the omega-3 dose is adjusted for the woman's height and the woman's age. In some embodiments, the omega-3 dose is adjusted for the woman's height and the woman's current weight. In some embodiments, the omega-3 dose is adjusted for the woman's height and the woman's pre-pregnancy weight. In some embodiments, the omega-3 dose is adjusted for the woman's height and the woman's medical history. In some embodiments, the omega-3 dose is adjusted for the woman's height and the woman's lifestyle. In some embodiments, the omega-3 dose is adjusted for the woman's height and the woman's genes. In some embodiments, the omega-3 dose is adjusted for the woman's height and the duration of the woman's current pregnancy. In some embodiments, the omega-3 dose is adjusted for the woman's height and the woman's race or ethnicity. In some embodiments, the omega-3 dose is adjusted for the woman's height and the woman's integrated socioeconomic status score.

In some embodiments, the omega-3 dose is adjusted for the woman's medical history and one or more additional parameters. In some embodiments, the omega-3 dose is adjusted for the woman's medical history and the woman's current omega-3 consumption. In some embodiments, the omega-3 dose is adjusted for the woman's medical history and the woman's age. In some embodiments, the omega-3 dose is adjusted for the woman's medical history and the woman's current weight. In some embodiments, the omega-3 dose is adjusted for the woman's medical history and the woman's pre-pregnancy weight. In some embodiments, the omega-3 dose is adjusted for the woman's medical history and the woman's height. In some embodiments, the omega-3 dose is adjusted for the woman's medical history and the woman's lifestyle. In some embodiments, the omega-3 dose is adjusted for the woman's medical history and the woman's genes. In some embodiments, the omega-3 dose is adjusted for the woman's medical history and the duration of the woman's current pregnancy. In some embodiments, the omega-3 dose is adjusted for the woman's medical history and the woman's race or ethnicity. In some embodiments, the omega-3 dose is adjusted for the woman's medical history and the woman's integrated socioeconomic status score.

In some embodiments, the omega-3 dose is adjusted for the woman's lifestyle and one or more additional parameters. In some embodiments, the omega-3 dose is adjusted for the woman's lifestyle and the woman's current omega-3 consumption. In some embodiments, the omega-3 dose is adjusted for the woman's lifestyle and the woman's age. In some embodiments, the omega-3 dose is adjusted for the woman's lifestyle and the woman's current weight. In some embodiments, the omega-3 dose is adjusted for the woman's lifestyle and the woman's pre-pregnancy weight. In some embodiments, the omega-3 dose is adjusted for the woman's lifestyle and the woman's height. In some embodiments, the omega-3 dose is adjusted for the woman's lifestyle and the woman's medical history. In some embodiments, the omega-3 dose is adjusted for the woman's lifestyle and the woman's genes. In some embodiments, the omega-3 dose is adjusted for the woman's lifestyle and the duration of the woman's current pregnancy. In some embodiments, the omega-3 dose is adjusted for the woman's lifestyle and the woman's race or ethnicity. In some embodiments, the omega-3 dose is adjusted for the woman's lifestyle and the woman's integrated socioeconomic status score.

In some embodiments, the omega-3 dose is adjusted for the woman's genes and one or more additional parameters. In some embodiments, the omega-3 dose is adjusted for the woman's genes and the woman's current omega-3 consumption. In some embodiments, the omega-3 dose is adjusted for the woman's genes and the woman's age. In some embodiments, the omega-3 dose is adjusted for the woman's genes and the woman's current weight. In some embodiments, the omega-3 dose is adjusted for the woman's genes and the woman's pre-pregnancy weight. In some embodiments, the omega-3 dose is adjusted for the woman's genes and the woman's height. In some embodiments, the omega-3 dose is adjusted for the woman's genes and the woman's medical history. In some embodiments, the omega-3 dose is adjusted for the woman's genes and the woman's lifestyle. In some embodiments, the omega-3 dose is adjusted for the woman's genes and the duration of the woman's current pregnancy. In some embodiments, the omega-3 dose is adjusted for the woman's genes and the woman's race or ethnicity. In some embodiments, the omega-3 dose is adjusted for the woman's genes and the woman's integrated socioeconomic status score.

In some embodiments, the omega-3 dose is adjusted for the duration of the woman's current pregnancy and one or more additional parameters. In some embodiments, the omega-3 dose is adjusted for the duration of the woman's current pregnancy and the woman's current omega-3 consumption. In some embodiments, the omega-3 dose is adjusted for the duration of the woman's current pregnancy and the woman's age. In some embodiments, the omega-3 dose is adjusted for the duration of the woman's current pregnancy and the woman's current weight. In some embodiments, the omega-3 dose is adjusted for the duration of the woman's current pregnancy and the woman's pre-pregnancy weight. In some embodiments, the omega-3 dose is adjusted for the duration of the woman's current pregnancy and the woman's height. In some embodiments, the omega-3 dose is adjusted for the duration of the woman's current pregnancy and the woman's medical history. In some embodiments, the omega-3 dose is adjusted for the duration of the woman's current pregnancy and the woman's lifestyle. In some embodiments, the omega-3 dose is adjusted for the duration of the woman's current pregnancy and the woman's genes. In some embodiments, the omega-3 dose is adjusted for the duration of the woman's current pregnancy and the woman's race or ethnicity. In some embodiments, the omega-3 dose is adjusted for the duration of the woman's current pregnancy and the woman's integrated socioeconomic status score.

In some embodiments, the omega-3 dose is adjusted for the woman's race or ethnicity and one or more additional parameters. In some embodiments, the omega-3 dose is adjusted for the woman's race or ethnicity and the woman's current omega-3 consumption. In some embodiments, the omega-3 dose is adjusted for the woman's race or ethnicity and the woman's age. In some embodiments, the omega-3 dose is adjusted for the woman's race or ethnicity and the woman's current weight. In some embodiments, the omega-3 dose is adjusted for the woman's race or ethnicity and the woman's pre-pregnancy weight. In some embodiments, the omega-3 dose is adjusted for the woman's race or ethnicity and the woman's height. In some embodiments, the omega-3 dose is adjusted for the woman's race or ethnicity and the woman's medical history. In some embodiments, the omega-3 dose is adjusted for the woman's race or ethnicity and the woman's lifestyle. In some embodiments, the omega-3 dose is adjusted for the woman's race or ethnicity and the woman's genes. In some embodiments, the omega-3 dose is adjusted for the woman's race or ethnicity and the duration of the woman's current pregnancy. In some embodiments, the omega-3 dose is adjusted for the woman's race or ethnicity and the woman's integrated socioeconomic status score.

In some embodiments, the omega-3 dose is adjusted for the woman's integrated socioeconomic status score and one or more additional parameters. In some embodiments, the omega-3 dose is adjusted for the woman's integrated socioeconomic status score and the woman's current omega-3 consumption. In some embodiments, the omega-3 dose is adjusted for the woman's integrated socioeconomic status score and the woman's age. In some embodiments, the omega-3 dose is adjusted for the woman's integrated socioeconomic status score and the woman's current weight. In some embodiments, the omega-3 dose is adjusted for the woman's integrated socioeconomic status score and the woman's pre-pregnancy weight. In some embodiments, the omega-3 dose is adjusted for the woman's integrated socioeconomic status score and the woman's height. In some embodiments, the omega-3 dose is adjusted for the woman's integrated socioeconomic status score and the woman's medical history. In some embodiments, the omega-3 dose is adjusted for the woman's integrated socioeconomic status score and the woman's lifestyle. In some embodiments, the omega-3 dose is adjusted for the woman's integrated socioeconomic status score and the woman's genes. In some embodiments, the omega-3 dose is adjusted for the woman's integrated socioeconomic status score and the duration of the woman's current pregnancy. In some embodiments, the omega-3 dose is adjusted for the woman's integrated socioeconomic status score and the woman's race or ethnicity.

### Omega-3 Supplementation Dose

As used herein, an “omega-3 supplementation dose” is a dose of omega-3 fatty acids which is intended to increase the woman's omega-3 level.

The omega-3 supplementation dose may provide from 300 to 1000 mg omega-3 fatty acids per day. For example, the omega-3 supplementation dose may provide 262.5 to 875 mg DHA per day and/or 37.5 to 125 mg EPA per day. Suitably, the omega-3 supplementation dose may provide from 600 to 1000 mg omega-3 fatty acids per day. For example, the omega-3 supplementation dose may provide 525 to 875 mg DHA per day and/or 75 to 125 mg EPA per day. Suitably, the omega-3 supplementation dose may provide from 700 to 900 mg omega-3 fatty acids per day or about 800 mg omega-3 fatty acids per day. For example, the omega-3 supplementation dose may provide 612.5 to 787.5 mg DHA per day and/or 97.5 to 112.5 mg EPA per day or about 700 mg DHA per day and/or about 100 mg EPA per day.

Suitably, the omega-3 supplementation dose may provide from 640 to 960 mg DHA per day and/or from 80 to 120 mg EPA per day. Suitably, the omega-3 supplementation dose may provide from 720 to 880 mg DHA per day and/or from 90 to 110 mg EPA per day. Suitably, the omega-3 supplementation dose may provide about 800 mg DHA per day and/or about 100 mg EPA per day.

The omega-3 supplementation dose may be adjusted for the one or more additional parameters. For example, the omega-3 supplementation dose may be adjusted for the woman's current omega-3 consumption, the woman's age, the woman's current weight, the woman's pre-pregnancy weight, the woman's height, the woman's medical history, the woman's lifestyle, the woman's genes, the duration of the woman's current pregnancy, the woman's race or ethnicity, and the woman's integrated socioeconomic status score.

In some embodiments, the omega-3 supplementation dose is adjusted for the woman's current omega-3 consumption and, optionally, one or more additional parameters. In some embodiments, the omega-3 supplementation dose is adjusted for the woman's age and, optionally, one or more additional parameters. In some embodiments, the omega-3 supplementation dose is adjusted for the woman's current weight and, optionally, one or more additional parameters. In some embodiments, the omega-3 supplementation dose is adjusted for the woman's pre-pregnancy weight and, optionally, one or more additional parameters. In some embodiments, the omega-3 supplementation dose is adjusted for the woman's height and, optionally, one or more additional parameters. In some embodiments, the omega-3 supplementation dose is adjusted for the woman's medical history and, optionally, one or more additional parameters. In some embodiments, the omega-3 supplementation dose is adjusted for the woman's lifestyle and, optionally, one or more additional parameters. In some embodiments, the omega-3 supplementation dose is adjusted for the woman's genes and, optionally, one or more additional parameters. In some embodiments, the omega-3 supplementation dose is adjusted for the duration of the woman's current pregnancy and, optionally, one or more additional parameters. In some embodiments, the omega-3 supplementation dose is adjusted for the woman's race or ethnicity and, optionally, one or more additional parameters. In some embodiments, the omega-3 supplementation dose is adjusted for the woman's integrated socioeconomic status score and, optionally, one or more additional parameters.

### Omega-3 Maintenance Dose

As used herein, an “omega-3 maintenance dose” is a dose of omega-3 fatty acids which is intended to maintain the woman's omega-3 level.

The omega-3 maintenance dose may provide from 100 to 300 mg omega-3 fatty acids per day or from 100 to less than 300 mg omega-3 fatty acids per day. For example, the omega-3 maintenance dose may provide 87.5 to 262.5 mg DHA per day and/or 12.5 to 37.5 mg EPA per day; or 87.5 to less than 262.5 mg DHA per day and/or 12.5 to less than 37.5 mg EPA per day. Suitably, the omega-3 maintenance dose may provide from 150 to 250 mg omega-3 fatty acids per day or about 200 mg omega-3 fatty acids per day. For example, the omega-3 maintenance dose may provide 130 to 220 mg DHA per day and/or 15 to 35 mg EPA per day or about 175 mg DHA per day and/or about 25 mg EPA per day.

Suitably, the omega-3 maintenance dose may provide from 160 to 240 mg DHA per day and/or from 20 to 30 mg EPA per day. Suitably, the omega-3 maintenance dose may provide from 180 to 220 mg DHA per day and/or from 22.5 to 27.5 mg EPA per day. Suitably, the omega-3 supplementation dose may provide about 200 mg DHA per day and/or about 25 mg EPA per day.

The omega-3 maintenance dose may be adjusted for the one or more additional parameters. For example, the omega-3 maintenance dose may be adjusted for the woman's current omega-3 consumption, the woman's age, the woman's current weight, the woman's pre-pregnancy weight, the woman's height, the woman's medical history, the woman's lifestyle, the woman's genes, the duration of the woman's current pregnancy, the woman's race or ethnicity, and the woman's integrated socioeconomic status score.

In some embodiments, the omega-3 maintenance dose is adjusted for the woman's current omega-3 consumption and, optionally, one or more additional parameters. In some embodiments, the omega-3 maintenance dose is adjusted for the woman's age and, optionally, one or more additional parameters. In some embodiments, the omega-3 maintenance dose is adjusted for the woman's current weight and, optionally, one or more additional parameters. In some embodiments, the omega-3 maintenance dose is adjusted for the woman's pre-pregnancy weight and, optionally, one or more additional parameters. In some embodiments, the omega-3 maintenance dose is adjusted for the woman's height and, optionally, one or more additional parameters. In some embodiments, the omega-3 maintenance dose is adjusted for the woman's medical history and, optionally, one or more additional parameters. In some embodiments, the omega-3 maintenance dose is adjusted for the woman's lifestyle and, optionally, one or more additional parameters. In some embodiments, the omega-3 maintenance dose is adjusted for the woman's genes and, optionally, one or more additional parameters. In some embodiments, the omega-3 maintenance dose is adjusted for the duration of the woman's current pregnancy and, optionally, one or more additional parameters. In some embodiments, the omega-3 maintenance dose is adjusted for the woman's race or ethnicity and, optionally, one or more additional parameters. In some embodiments, the omega-3 maintenance dose is adjusted for the woman's integrated socioeconomic status score and, optionally, one or more additional parameters.

### Omega-3 Low Dose

As used herein, an “omega-3 low dose” is a dose of omega-3 fatty acids which is not intended to increase the woman's omega-3 level. For example, an omega-3 low dose may be a dose of omega-3 fatty acids which is intended to decrease the woman's omega-3 level. Suitably, the omega-3 low dose may comprise trace amounts of omega-3 fatty acids.

The omega-3 low dose may provide less than 100 mg omega-3 fatty acids per day. For example, the omega-3 low dose may provide less than 87.5 mg DHA per day and/or less than 12.5 mg EPA per day. Suitably, the omega-3 low dose may provide less than 80 mg omega-3 fatty acids per day, less than 40 mg omega-3 fatty acids per day, or less than 20 mg omega-3 fatty acids per day. For example, the omega-3 low dose may provide less than 70 mg DHA per day and/or less than 10 mg EPA per day, or less than 35 mg DHA per day and/or less than 5 mg EPA per day, or less than 17.5 mg DHA per day and/or less than 2.5 mg EPA per day.

Suitably, the omega-3 low dose may provide about 15 mg DHA per day or less and/or about 4 mg EPA per day or less.

The omega-3 low dose may be adjusted for the one or more additional parameters. For example, the omega-3 low dose may be adjusted for the woman's current omega-3 consumption, the woman's age, the woman's current weight, the woman's pre-pregnancy weight, the woman's height, the woman's medical history, the woman's lifestyle, the woman's genes, the duration of the woman's current pregnancy, the woman's race or ethnicity, and the woman's integrated socioeconomic status score.

In some embodiments, the omega-3 low dose is adjusted for the woman's current omega-3 consumption and, optionally, one or more additional parameters. In some embodiments, the omega-3 low dose is adjusted for the woman's age and, optionally, one or more additional parameters. In some embodiments, the omega-3 low dose is adjusted for the woman's current weight and, optionally, one or more additional parameters. In some embodiments, the omega-3 low dose is adjusted for the woman's pre-pregnancy weight and, optionally, one or more additional parameters. In some embodiments, the omega-3 low dose is adjusted for the woman's height and, optionally, one or more additional parameters. In some embodiments, the omega-3 low dose is adjusted for the woman's medical history and, optionally, one or more additional parameters. In some embodiments, the omega-3 low dose is adjusted for the woman's lifestyle and, optionally, one or more additional parameters. In some embodiments, the omega-3 low dose is adjusted for the woman's genes and, optionally, one or more additional parameters. In some embodiments, the omega-3 low dose is adjusted for the duration of the woman's current pregnancy and, optionally, one or more additional parameters. In some embodiments, the omega-3 low dose is adjusted for the woman's race or ethnicity and, optionally, one or more additional parameters. In some embodiments, the omega-3 low dose is adjusted for the woman's integrated socioeconomic status score and, optionally, one or more additional parameters.

The omega-3 low dose may provide a multivitamin and/or mineral supplement.

### Omega-3 Zero Dose

As used herein, an “omega-3 zero dose” is a dose of omega-3 fatty acids which comprises negligible, non-detectable, or zero amounts of omega-3 fatty acids.

The omega-3 zero dose may provide less than 10 mg omega-3 fatty acids per day. Suitably, the omega-3 zero dose may provide less than 5 mg omega-3 fatty acids per day, less than 1 mg omega-3 fatty acids per day or about 0 mg omega-3 fatty acids per day.

The omega-3 zero dose may provide a multivitamin and/or mineral supplement.

### Thresholds and Risk of Early Preterm Birth

The woman's personalised omega-3 dose may be determined by any suitable method using the woman's omega-3 level, and optionally the one or more additional parameters.

Typically, the personalised omega-3 dose will vary depending on the woman's omega-3 level, and optionally the one or more additional parameters. For example, when the woman's omega-3 level is low the personalised omega-3 dose may be an omega-3 supplementation dose; when the woman's omega-3 level is adequate the personalised omega-3 dose may be an omega-3 maintenance dose; when the woman's omega-3 level is high the personalised omega-3 dose may be an omega-3 low dose or zero dose.

One or more thresholds may be used to define two or more omega-3 doses. Suitably, one or more (e.g. 1, 2, 3, 4, 5, 6, 7, 8) thresholds are used. Preferably, two or more e.g. 2, 3, 4, 5, 6, 7, 8) thresholds are used. More preferably, two thresholds are used. Suitably, two or more e.g. 2, 3, 4, 5, 6, 7, 8) doses are defined. Preferably, three or more e.g. 3, 4, 5, 6, 7, 8) doses are defined. More preferably, three doses are defined. Suitably, (i) if the omega-3 level is equal to or below a first threshold, the personalised omega-3 dose is a first omega-3 dose; and (ii) if the omega-3 level is above the first threshold, the personalised omega-3 dose is a second omega-3 dose. Suitably, (i) if the omega-3 level is equal to or below a first threshold, the personalised omega-3 dose is a first omega-3 dose; (ii) if the omega-3 level is above the first threshold but below a second threshold, the personalised omega-3 dose is a second omega-3 dose; and (iii) if the omega-3 level is equal to or above the second threshold, the personalised omega-3 dose is a third omega-3 dose.

In some embodiments:
(i) if the omega-3 level is equal to or below a first threshold, the personalised omega-3 dose is an omega-3 supplementation dose;
(ii) if the omega-3 level is above the first threshold but below a second threshold the personalised omega-3 dose is an omega-3 maintenance dose; and/or
(iii) if the omega-3 level is equal to or above the second threshold the personalised omega-3 dose is an omega-3 low dose or an omega-3 zero dose.

The thresholds may be given in any suitable measurement unit and to any suitable precision. Suitably, the thresholds are given in the total amount of omega-3 fatty acids as a percentage of total fatty acids in the woman's blood and/or the precision is to one decimal place.

The thresholds may be determined by any suitable method. Suitably, the thresholds are adjusted for the one or more additional parameters. For example, the thresholds may be adjusted for women with pre-pregnancy obesity because it is known that maternal pre-pregnancy obesity attenuates response to omega-3 fatty acids supplementation during pregnancy (Monthé-Drèze, C., et al., 2018. Nutrients, 10(12), p. 190). For example, the thresholds may be adjusted for women with genetic variants of the FADS1 FADS2 gene cluster because it is known that genetic variants of the FADS1 FADS2 gene cluster are associated with altered (n-3) essential fatty acids in plasma in women during pregnancy (Xie, L. and Innis, S. M., 2008. The Journal of nutrition, 138(11), pp. 2222-2228).

Any suitable method may be used to adjust the threshold for the one or more additional parameters. For example, multivariate log-binomial regression models adjusted by the additional parameters may be applied to estimate the relative risk of maternal or infant outcomes due to omega-3 supplementation varied by baseline omega-3 status and the thresholds may be adjusted accordingly. Suitably, the thresholds are adjusted for the one or more additional parameters using a multivariate regression model.

Suitably, the first threshold and/or the second threshold are determined using the woman's risk of early preterm birth (R EPTB). The woman's R EPTB is the probability of the woman having an early preterm birth (EPTB). The R EPTB may be determined by any statistical method known in the art. For example, trials in which the omega-3 level, and optionally the one or more additional parameters, and the incidence of EPTB is measured for a cohort of pregnant women can be used to estimate the woman's R EPTB.

Suitably, the first threshold and/or the second threshold are determined using the woman's relative risk of early preterm birth (RR EPTB). The woman's RR EPTB is the ratio of the probability of the woman having an EPTB when given a selected omega-3 dose (e.g. an omega-3 supplementation dose) to the probability of the woman having an EPTB when given an omega-3 low dose or omega-3 zero dose (e.g. an omega-3 zero dose). The woman's RR EPTB may be determined by any suitable statistical method known in the art (see e.g. Coggon, D., Barker, D. and Rose, G., 2009. Epidemiology for the Uninitiated, Chapter 3. John Wiley & Sons). For example, trials in which the omega-3 level, and optionally the one or more additional parameters, and the incidence of EPTB is measured for a cohort of pregnant women who have taken a selected omega-3 dose (e.g. an omega-3 supplementation dose) and for a cohort of pregnant women who have taken an omega-3 low dose or omega-3 zero dose (e.g. an omega-3 zero dose) can be used to estimate the woman's RR EPTB.

The woman's RR EPTB may be determined using the woman's omega-3 level and, optionally, the one or more additional parameters described herein. For example, the woman's RR EPTB may be determined using the woman's omega-3 level and adjusted for the one or more additional parameters.

Any suitable method may be used to adjust the woman's RR EPTB for the one or more additional parameters. For example, multivariate log-binomial regression models adjusted by the additional parameters may be applied to estimate the relative risk of maternal or infant outcomes due to omega-3 supplementation varied by baseline omega-3 status and the RR EPTB may be adjusted accordingly. Suitably, the omega-3 dose is adjusted for the one or more additional parameters using a multivariate regression model. Suitably, the woman's RR EPTB is adjusted for the one or more additional parameters using a multivariate regression model.

The woman's RR EPTB may be determined using a regression equation. For example, the method of the present invention may comprise determining the woman's RR EPTB using a regression equation relating the woman's RR EPTB to the woman's omega-3 level and, optionally, one or more additional parameters described herein.

As used herein, a “regression equation” relates a dependent variable (e.g. woman's RR EPTB) to one or more independent variables (e.g. woman's omega-3 level). In some embodiments, the regression equation is a multiple regression equation. As used herein, a “multiple regression equation” relates a dependent variable to two or more independent variables. In some embodiments, the regression equation is a linear regression equation. A “linear regression equation” has an equation of the form Y=a+bX, where X is the independent variable and Y is the dependent variable. The slope of the line is b, and a is the intercept. In some embodiments, the regression equation is a multiple linear regression equation. A “multiple linear regression equation” has an equation of the form Y=a+b1X1+b2X2+b3X3+ . . . +bnXn, where Xn is an independent variable and Y is the dependent variable. The regression coefficient for each independent variable is bn, and a is the intercept.

Each of the intercept and coefficients can be readily determined using any statistical method known in the art. For example, the intercept and coefficients may be determined by multiple linear regression. Such routine statistical methods may include multiple linear regression with bootstrapping. It is possible to obtain the same estimates with generalised linear or additive models or any other regression-related model with various estimation algorithms, for example, elastic net, lasso, Bayesian approach etc. The one or more parameters may be transformed prior to using as a variable in the regression equation. Any suitable transformation may be used. For example, a log transformation may be used to address the skewed distribution of a parameter. Any measurement unit may be used.

A RR EPTB<1 means that the selected omega-3 dose (e.g. an omega-3 supplementation dose) reduces the risk of EPTB. A RR EPTB=1 means that the selected omega-3 dose (e.g. an omega-3 supplementation dose) does not affect the risk of EPTB. A RR EPTB>1 means that the selected omega-3 dose (e.g. an omega-3 supplementation dose) increases the risk of EPTB. The first threshold may be an omega-3 level at and/or below which the woman's RR EPTB is less than one or significantly less than one. The second threshold may be an omega-3 level at and/or above which the woman's RR EPTB is one or significantly more than one.

As used herein “significantly” different may mean that the difference is statistically significant. Suitably, “significantly” less or more may mean that the 90% confidence interval, the 95% confidence interval, the 98% confidence interval, or the 99% confidence interval is less than or greater than the specified value, respectively. Preferably, “significantly” less or more means that the 95% confidence interval is less than or greater than the specified value, respectively. For example, the woman's RR EPTB may be significantly less than one if the 95% confidence interval is less than one and/or the woman's RR EPTB may be significantly more than one if the 95% confidence interval is more than one.

In some embodiments, the first threshold is an omega-3 level of 4.5% or less, 4.4% or less, 4.3% or less, 4.2% or less, or 4.1% or less as a percentage of total fatty acids in the woman's blood. In some embodiments, the first threshold is an omega-3 level of 3.5% or more, 3.6% or more, 3.7% or more, 3.8% or more, 3.9% or more, 4.0% or more, or 4.1% or more as a percentage of total fatty acids in the woman's blood. In some embodiments, the first threshold is an omega-3 level of from 3.5% to less than 4.5%, from 3.7% to 4.4%, from 3.9% to 4.3%, from 4.0% to 4.2%, or about 4.1% as a percentage of total fatty acids in the woman's blood.

In some embodiments, the second threshold is an omega-3 level of more than 4.5%, 4.6% or more, 4.7% or more, 4.8% or more, or 4.9% or more as a percentage of total fatty acids in the woman's blood. In some embodiments, the second threshold is an omega-3 level of 5.5% or less, 5.4% or less, 5.3% or less, 5.2% or less, 5.1% or less, 5.0% or less, or 4.9% or less as a percentage of total fatty acids in the woman's blood. In some embodiments, the second threshold is an omega-3 level of from more than 4.5% to 5.5%, from 4.6% to 5.3%, from 4.7% to 5.1%, from 4.8% to 5.0%, or about 4.9% as a percentage of total fatty acids in the woman's blood.

### Method for Providing a Personalised Omega-3 Dose

In one aspect, the present invention provides a method for providing a personalised omega-3 dose to be taken by a pregnant woman.

In one aspect, the present invention provides for use of a personalised omega-3 dose to be taken by a pregnant woman.

### Omega-3 Fatty Acids Supplement

The personalised omega-3 dose may be determined by any method described herein. The method may comprise determining the woman's omega-3 level.

The personalised omega-3 dose may be taken or administered at any time before or during pregnancy. Suitably, administration may start prior to 20 weeks of gestation, preferably administration starts at 12 to 16 weeks of gestation, more preferably administration starts at about 14 weeks of gestation. Suitably, the personalised omega-3 dose may be administered until about 34 weeks of gestation. Suitably, the personalised omega-3 dose is administered from before about 20 weeks of gestation to about 34 weeks of gestation, preferably the personalised omega-3 dose is administered from about 14 weeks of gestation (e.g. 12-16 weeks) to about 34 weeks of gestation.

The personalised omega-3 dose may be taken or administered in any suitable form, e.g. in a form suitable for oral or parenteral administration. Suitably, the personalised omega-3 dose is in a form suitable for oral administration. The personalised omega-3 dose may be taken or administered in the form of an omega-3 fatty acids supplement.

Suitably, the omega-3 fatty acids taken or administered are omega-3 LCPUFA. Suitably, the omega-3 fatty acids comprise docosahexaenoic acid (DHA) and eicosapentaenoic acid (EPA).

Docosahexaenoic acid (DHA) (also known as Doconexent, cervonic acid and (4Z,7Z,10Z, 13Z, 16Z, 19Z)-4,7,10,13,16, 19-Docosahexaenoic acid) is an omega-3 fatty acid with the following structure:

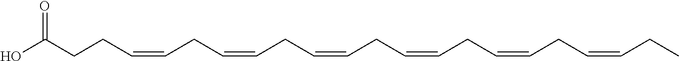 [SMILES: CC/C=C\C/C=C\C/C=C\C/C=C\C/C=C\C/C=C\CCC(=O)O]

Eicosapentaenoic acid (EPA) (also known as icosapentaenoic acid, timnodonic acid and (5Z,8Z,11Z,14Z, 17Z)-icosa-5,8,11,14,17-pentaenoic acid) is an omega-3 fatty acid with the following structure:

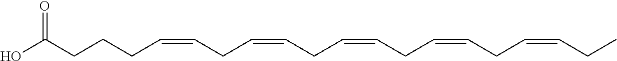 [SMILES: CC/C=C\C/C=C\C/C=C\C/C=C\C/C=C\CCCC(=O)O]

The omega-3 fatty acids may be derived from any source, for example fish oil and/or algal oil. For example, the omega-3 fatty acids supplement may be a fish oil supplement or an algal oil supplement as described in Ryan, L. and Symington, A. M., 2015. Journal of functional foods, 19, pp. 852-858.

A “supplement” or “dietary supplement” may be used to complement the nutrition of an individual (it is typically used as such but it might also be added to any kind of compositions intended to be ingested).

The omega-3 fatty acids supplement may be provided in the form of unit doses. For example, each unit dose may comprise about 50 mg, about 100 mg, or about 200 mg omega-3 fatty acids. In some embodiments, each unit dose comprises about 200 mg omega-3 fatty acids.

The omega-3 fatty acids supplement may be in the form of, for example, tablets, capsules, pastilles or a liquid. Suitably, the omega-3 fatty acids supplement is in the form of a capsule, for example a gelatin capsule.

The omega-3 fatty acids supplement may comprise DHA, EPA and other fatty acids. For example, the omega-3 fatty acids supplement may comprise other poly-unsaturated fatty acids (PUFAs) such as docosapentaenoic acid (DPA) and the like. For example, a fish oil supplement may comprise about 22% DHA and 30% EPA by weight total fatty acids (as described in Barrow, C. J., et al., 2009. Journal of Functional Foods, 1(1), pp. 38-43). For example, an algal oil supplement may comprise about 40% DHA, 15% DPA and 2.5% EPA by weight total fatty acids (as described in Arterburn, L. M., et al., 2007. Lipids, 42(11), p. 1011).

The omega-3 fatty acids supplement may comprise from 60% to 80% by weight omega-3 fatty acids. The omega-3 fatty acids supplement may comprise 50% to 70% by weight DHA and/or 5% to 15% by weight EPA.

The omega-3 fatty acids supplement may contain an organic or inorganic carrier material suitable for oral or parenteral administration as well as vitamins, minerals trace elements and other micronutrients.

### Promoting Full-Term Birth

The personalised omega-3 dose may promote a full-term birth and/or reduce the risk or a preterm and/or early preterm birth.

The personalised omega-3 dose may promote a full-term birth. As used herein “promote a full-term birth” means that taking the personalised omega-3 dose may increase the probability of the pregnant woman having a full-term birth compared to taking a different omega-3 dose (e.g. an omega-3 dose which differs by at least 100 mg, at least 200 mg, or at least 400 mg omega-3 fatty acids per day from the personalised omega-3 dose). Full-term birth (FTB) may include early term birth and be defined as birth from 37 weeks of gestation e.g. from 37 weeks to before 41 weeks of gestation. Full-term birth (FTB) may also be defined as birth from 39 weeks of gestation e.g. from 39 weeks to before 41 weeks of gestation.

The personalised omega-3 dose may reduce the risk or a preterm and/or early preterm birth. As used herein “reduce the risk of a preterm and/or early preterm birth” means that taking the personalised omega-3 dose may decrease the probability of the pregnant woman having a preterm and/or early preterm birth compared to taking a different omega-3 dose (e.g. an omega-3 dose which differs by at least 100 mg, at least 200 mg, or at least 400 mg omega-3 fatty acids per day from the personalised omega-3 dose). Preterm birth (PTB) may be defined as birth before about 37 weeks of gestation and early PTB (EPTB) may be defined as birth before about 34 weeks of gestation.

### Computer Program and Computer-Readable Medium

The method described herein for determining a personalised omega-3 dose to be taken by a pregnant woman may be a computer-implemented method.

In one aspect, the present invention provides a computer-implemented method for determining a personalised omega-3 dose to be taken by a pregnant woman to promote a full-term birth, wherein the method comprises using the woman's omega-3 level to determine the personalised omega-3 dose

In one aspect, the present invention provides a data processing system comprising means for determining a personalised omega-3 dose to be taken by a pregnant woman to promote a full-term birth, given the woman's omega-3 level and optionally one or more additional parameters.

In one aspect, the present invention provides a data processing apparatus comprising a processor configured to determine a personalised omega-3 dose to be taken by a pregnant woman to promote a full-term birth, given the woman's omega-3 level and optionally one or more additional parameters.

In one aspect, the present invention provides a computer program comprising instructions which, when the program is executed by a computer, cause the computer to determine a personalised omega-3 dose to be taken by a pregnant woman to promote a full-term birth, given the woman's omega-3 level and optionally one or more additional parameters.

In one aspect, the present invention provides a computer-readable medium comprising instructions which, when executed by a computer, cause the computer to determine a personalised omega-3 dose to be taken by a pregnant woman to promote a full-term birth, given the woman's omega-3 level and optionally one or more additional parameters.

The additional parameters may be any additional parameters described herein. The personalised omega-3 dose may be determined by any method described herein.

In one aspect, the present invention provides a data processing system comprising means for carrying out the method of the invention.

In one aspect, the present invention provides a data processing apparatus comprising a processor configured to perform the method of the invention.

In one aspect, the present invention provides a computer program comprising instructions which, when the program is executed by a computer, cause the computer to carry out the method of the invention.

In one aspect, the present invention provides a computer-readable medium comprising instructions which, when executed by a computer, cause the computer to carry out the method of the invention.

In one aspect, the present invention provides a computer-readable data carrier having stored thereon the computer program of the invention.

In one aspect, the present invention provides a data carrier signal carrying the computer program of the invention.

### EXAMPLES

The invention will now be further described by way of examples, which are meant to serve to assist the skilled person in carrying out the invention and are not intended in any way to limit the scope of the invention.

### Example 1—Developing an Algorithm to Determine a Personalised Omega-3 Dose

### Algorithm to Determine a Personalised Omega-3 Dose

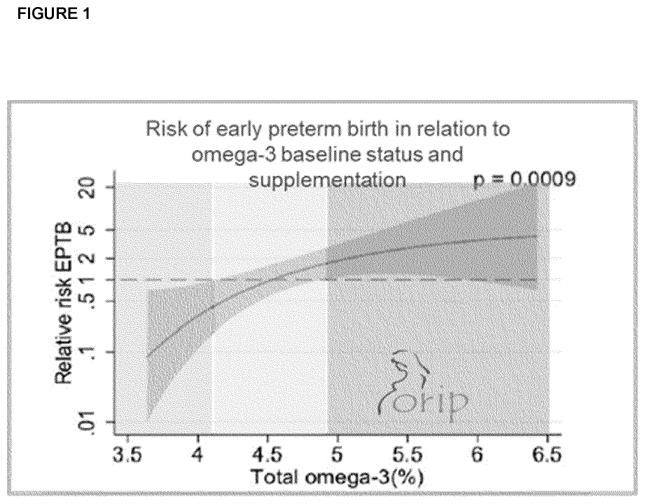

The published data of the Omega-3 to Reduce the Incidence of Prematurity (ORIP) trial was used to develop an algorithm to determine a personalised omega-3 dose. ORIP gives insight on how supplementation may affect the risk of early preterm birth (EPTB). An exemplary algorithm is as follows:
i) Blood sample: dry blood spot
ii) Baseline stage: before 20 weeks of pregnancy
iii) Biomarkers: total omega-3 PUFA (DHA+EPA+DPA+ALA)
iv) Rules:
   (1) If baseline total omega-3≤ 4.1% (left-hand zone in FIG. 1), 800 mg DHA+100 mg EPA provided
   (2) If baseline total omega-3>4.1% and ≤4.9% (middle zone in FIG. 1), 200 mg DHA+25 mg EPA provided
   (3) If baseline total omega-3>4.9% (right-hand zone in FIG. 1), multivitamin provided

In order to provide personalized omega-3 dose, the following information may be obtained from the consumers (in addition to collection of dry blood spot sample):
1) Mother birth date (Day/Month/Year)
2) Mother weight
3) Mother height
4) EDD
5) Parity
6) Mode of conception (ART or normal)

A real-world evidence study may provide more granulated details, which may enhance the performances of the algorithm shown above. This also applies to the supplementary data (e.g., genetic data, dietary data, lifestyle data, etc.).

Heterogeneity between countries and population behaviors is observed in the literature. Taking into account the country/region heterogeneity at the deployment stage and conducting comparative analysis may enhance the model performance and serve local audiences better.

### Literature Review

The ORIP trial was a multicenter, double-blind, randomized, controlled clinical trial and was funded by the Australian National Health and Medical Research Council and the Thyne Reid Foundation. Its Clinical Trial Registry Number is 2613001142729. The trial protocol has been published. Women who were less than 20 weeks of pregnancy were randomized to receive high DHA fish oil capsules (approximately 800 mg of DHA and approximately 100 mg of EPA per day) or control capsules (approximately 15 mg of DHA and 4 mg of EPA per day), from enrolment until 34 weeks of gestation. A total of 2770 pregnancies in 2766 women were assigned to the study group and 2774 pregnancies in 2765 women to the control group. The compositions of the study capsules are shown in Table 1.

TABLE 1
Compositions of the ORIP study capsules

| | | Intervention | Control |
|---|---|---|---|
| Fatty acid | Fatty acid name | % | % |
| Total saturates | | 2.8-6.1 | 34.2-35.0 |
| Total monounsaturates | | 8.0-13.0 | 41.2-41.3 |
| 18:2 ω6 | linoleic | 0.4-0.6 | 19.8-20.0 |
| 20:4 ω6 | arachidonic | 2.3-3.5 | 0.1 |
| 22:5 ω6 | docosapentaenoic | 4.1-4.4 | 0.1 |
| Total ω6 | | 8.1-8.9 | 20.0-20.2 |
| 18:3 ω3 | α-linolenic | 0.2-0.3 | 1.8-1.9 |
| 18:4 ω3 | octadecatetraenoic | 0.3-0.5 | 0.1 |
| 20:4 ω3 | eicosatetraenoic | 0.3-0.7 | — |
| 20:5 ω3 | eicosapentaenoic | 8.0-11.7 | 0.3 |
| 22:5 ω3 | docosapentaenoic | 2.3-2.6 | — |
| 22:6 ω3 | docosahexaenoic | 55.9-58.8 | 1.0-1.1 |
| Total ω3 | | 68.2-74.0 | 3.2-3.4 |
| Minor components | | 3.0-6.4 | 0.6-0.9 |

The primary outcome was the incidence of EPTB (early preterm birth), defined as delivery before 34 completed weeks of gestation.

The proportion of total omega-3 (in the total fatty acids in maternal capillary blood) was identified as the best biomarker to distinguish which women with singleton pregnancies are at increased risk of EPTB. As shown in FIG. 1, Women with total omega-3 baseline of 4.1% or below of total fatty acids are at higher risk of EPTB and are most likely to benefit from omega-3 supplementation to reduce this risk. Conversely, women with total omega-3 baseline above 4.9% of total fatty acids in early pregnancy are at lower risk of EPTB and additional omega-3 supplementation may increase their risk (Simmonds L. A., et al., 2020. BJOG: An International Journal of Obstetrics & Gynaecology, 127(8), pp. 975-981).

In addition to the ORIP trial mentioned above, several other studies with EPTB or PTB outcomes were also reviewed:
European Food Safety Authority (EFSA) panel on dietetic Products, nutrition, and allergies (NDA) suggest an additional intake of 100-200 mg DHA & EPA per day for pregnant and lactating women, on top of the recommendation of 250 mg DHA & EPA per day for health adults (EFSA J. 2013; 11:3408);
Carlson's study indicated achieving a score of 5% of total fatty acids or higher is optimal and anything below that number would increase the risk of preterm birth (Carlson, S. E., et al. (2017). BMC pregnancy and childbirth, 17(1), 62);
Middleton's Cochrane review summarized Women who took a DHA supplement during pregnancy were 42% less likely to have early preterm birth compared to those who did not (Middleton, P., et al. (2018). The Cochrane database of systematic reviews, 11(11), CD003402);
Olsen's study across various pregnancy stage showed establishing DHA status during the different stages of pregnancy could play a crucial role in preventing prematurity (Olsen, S. F., et al. (2018). EBioMedicine, 35, 325-333).

### EMBODIMENTS

Various preferred features and embodiments of the present invention will now be described with reference to the following numbered paragraphs (paras).

1. A method for determining a personalised omega-3 dose to be taken by a pregnant woman to promote a full-term birth, wherein the method comprises using the woman's omega-3 level to determine the personalised omega-3 dose.

2. The method according to para 1, wherein the method further comprises using one or more additional parameters to determine the personalised omega-3 dose.

3. The method according to para 2, wherein the one or more additional parameters comprise one or more parameter selected from: the woman's current omega-3 consumption, the woman's age, the woman's current weight, the woman's pre-pregnancy weight, the woman's height, the woman's medical history, the woman's lifestyle, the woman's genes, the duration of the woman's current pregnancy, the woman's race or ethnicity, and the woman's integrated socioeconomic status score.

4. The method according to para 2 or 3, wherein the one or more additional parameters comprise one or more parameter selected from: the woman's current omega-3 consumption, the woman's age, the woman's current weight, the woman's pre-pregnancy weight, the woman's height, the woman's medical history, the woman's lifestyle, and the woman's genes.

5. The method according to any of paras 2-4, wherein the one or more additional parameters comprise one or more parameter selected from: the woman's current omega-3 consumption, the woman's age, the woman's current weight, the woman's pre-pregnancy weight, and the woman's height.

6. The method according to any of paras 3-5, wherein the woman's medical history comprises one or more parameter selected from: pre-eclampsia or eclampsia in a previous pregnancy, previous pre-term birth, fertility treatment in current pregnancy, woman's compliance, type 2 diabetes, gestational diabetes mellitus in previous pregnancy, woman born preterm, woman's gravidity, woman's parity, any previous pregnancy ended before twenty weeks, previous stillbirth, and previous neonatal death.

7. The method according to any of paras 3-6, wherein the woman's medical history comprises one or more parameter selected from: pre-eclampsia or eclampsia in a previous pregnancy, previous pre-term birth, fertility treatment in current pregnancy, woman's compliance, type 2 diabetes, and gestational diabetes mellitus in previous pregnancy.

8. The method according to any of paras 3-7, wherein the woman's lifestyle comprises smoking status and/or alcohol consumption.

9. The method according to any preceding para, wherein:
(i) if the omega-3 level is equal to or below a first threshold, the personalised omega-3 dose is an omega-3 supplementation dose;
(ii) if the omega-3 level is above the first threshold but below a second threshold the personalised omega-3 dose is an omega-3 maintenance dose; and
(iii) if the omega-3 level is equal to or above the second threshold the personalised omega-3 dose is an omega-3 low dose or an omega-3 zero dose.

10. The method according to para 9, wherein the first threshold and the second threshold are determined using the woman's relative risk of early preterm birth (RR EPTB).

11. The method according to para 10, wherein the woman's RR EPTB is adjusted for the one or more additional parameters.

12. The method according to para 10 or 11, wherein the first threshold is an omega-3 level at and/or below which the woman's RR EPTB is significantly less than one.

13. The method according to any of paras 10 to 12, wherein the second threshold is an omega-3 level at and/or above which the woman's RR EPTB is significantly more than one.

14. The method according to para 12 or 13, wherein the woman's RR EPTB is significantly less than one if the 95% confidence interval is less than one, and wherein the woman's RR EPTB is significantly more than one if the 95% confidence interval is more than one.

15. The method according to any of paras 9 to 14, wherein the first threshold is an omega-3 level of less than 4.5%, 4.4% or less, 4.3% or less, 4.2% or less, or 4.1% or less.

16. The method according to any of paras 9 to 15, wherein the first threshold is an omega-3 level of 3.5% or more, 3.6% or more, 3.7% or more, 3.8% or more, 3.9% or more, 4.0% or more, or 4.1% or more.

17. The method according to any of paras 9 to 16, wherein the first threshold is an omega-3 level of from 3.5% to less than 4.5%, from 3.7% to 4.4%, from 3.9% to 4.3%, from 4.0% to 4.2%, or about 4.1%.

18. The method according to any of paras 9 to 17, wherein the second threshold is an omega-3 level of more than 4.5%, 4.6% or more, 4.7% or more, 4.8% or more, or 4.9% or more.

19. The method according to any of paras 9 to 18, wherein the second threshold is an omega-3 level of 5.5% or less, 5.4% or less, 5.3% or less, 5.2% or less, 5.1% or less, 5.0% or less, or 4.9% or less.

20. The method according to any of paras 9 to 19, wherein the second threshold is an omega-3 level of from more than 4.5% to 5.5%, from 4.6% to 5.3%, from 4.7% to 5.1%, from 4.8% to 5.0%, or about 4.9%.

21. The method according to any preceding para, wherein the omega-3 level is the total amount of omega-3 fatty acids as a percentage of total fatty acids in the woman's blood.

22. The method according to any preceding para, wherein the omega-3 fatty acids comprise or consist of docosahexaenoic acid (DHA), eicosapentaenoic acid (EPA), docosapentaenoic acid (DPA), and alpha-linolenic acid (ALA).

23. The method according to any preceding para, wherein the omega-3 level is determined prior to 20 weeks of gestation, preferably wherein the omega-3 level is determined at 12 to 16 weeks of gestation or about 14 weeks of gestation.

24. The method according to any preceding para, wherein the omega-3 level is determined using dried blood spot analysis, a home-based test kit, or conventional blood analysis, preferably dried spot analysis.

25. The method according to any preceding para, wherein the personalised omega-3 dose is a daily dose.

26. The method according to any of paras 9 to 25, wherein the omega-3 supplementation dose provides from 300 to 1000 mg omega-3 fatty acids per day, preferably wherein the omega-3 supplementation dose provides from 640 to 960 mg DHA per day and/or from 80 to 120 mg EPA per day, more preferably wherein the omega-3 supplementation dose provides about 800 mg DHA and about 100 mg EPA per day.

27. The method according to any of paras 9 to 26, wherein the omega-3 maintenance dose provides from 100 to 300 mg omega-3 fatty acids per day, preferably wherein the omega-3 maintenance dose provides from 160 to 240 mg DHA per day and/or from 20 to 30 mg EPA per day, more preferably wherein the omega-3 maintenance dose provides about 200 mg DHA and about 25 mg EPA per day.

28. The method according to any of paras 9 to 27, wherein the omega-3 low dose provides less than 100 mg omega-3 fatty acids per day.

29. The method according to any of paras 9 to 28, wherein the omega-3 low dose and/or the omega-3 zero dose provides a multivitamin and mineral supplement.

30. The method according to any of paras 9 to 29, wherein the omega-3 supplementation dose, omega-3 maintenance dose, omega-3 low dose, and/or omega-3 zero dose is adjusted for the one or more additional parameters.

31. The method according to any preceding para, wherein the method is a computer-implemented method.

32. A method for determining a personalised omega-3 dose to be taken by a pregnant woman to promote a full-term birth, wherein the method comprises:
(i) determining the woman's omega-3 level; and
(ii) determining the personalised omega-3 dose using the method according to any of paras 1 to 31.

33. A method for providing a personalised omega-3 dose to be taken by a pregnant woman to promote a full-term birth, wherein the method comprises:
(i) optionally, determining the woman's omega-3 level;
(ii) determining the personalised omega-3 dose using the method according to any of paras 1 to 31; and
(iii) administering the personalised omega-3 dose to the woman.

34. A method of promoting a full-term birth for a pregnant woman, the method comprising administering a personalised omega-3 dose to the woman, wherein the personalised omega-3 dose is determined using the method according to any of paras 1 to 31.

35. A computer program comprising instructions which, when the program is executed by a computer, cause the computer to carry out the method according to any of paras 1 to 31.

36. A computer program comprising instructions which, when the program is executed by a computer, cause the computer to determine a personalised omega-3 dose to be taken by a pregnant woman to promote a full-term birth, given the woman's omega-3 level and optionally one or more additional parameters.

37. A computer-readable medium comprising instructions which, when executed by a computer, cause the computer to carry out the method according to any of paras 1 to 31.

38. A computer-readable medium comprising instructions which, when executed by a computer, cause the computer to determine a personalised omega-3 dose to be taken by a pregnant woman to promote a full-term birth, given the woman's omega-3 level and optionally one or more additional parameters.

39. Use of a personalised omega-3 dose, wherein the personalised omega-3 dose is determined using the method according to any of paras 1 to 31.

All publications mentioned in the above specification are herein incorporated by reference. Various modifications and variations of the disclosed methods, compositions and uses of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

1. A method for determining a personalised omega-3 dose to be taken by a pregnant woman to promote a full-term birth, wherein the method comprises using the woman's omega-3 level to determine the personalised omega-3 dose.

2. The method according to claim 1, wherein the method further comprises using one or more additional parameters to determine the personalised omega-3 dose.

3. The method according to claim 2, wherein the one or more additional parameters comprise one or more parameter selected from the group consisting of: the woman's current omega-3 consumption, the woman's age, the woman's current weight, the woman's pre-pregnancy weight, the woman's height, the woman's medical history, the woman's lifestyle, the woman's genes, the duration of the woman's current pregnancy, the woman's race or ethnicity, and the woman's integrated socioeconomic status score.

4. The method according to claim 1, wherein:
(i) if the omega-3 level is equal to or below a first threshold, the personalised omega-3 dose is an omega-3 supplementation dose;
(ii) if the omega-3 level is above the first threshold but below a second threshold the personalised omega-3 dose is an omega-3 maintenance dose; and
(iii) if the omega-3 level is equal to or above the second threshold the personalised omega-3 dose is an omega-3 low dose or an omega-3 zero dose.

5. The method according to claim 4, wherein the first threshold and the second threshold are determined using the woman's relative risk of early preterm birth (RR EPTB), optionally wherein the woman's RR EPTB is adjusted for the one or more additional parameters.

6. The method according to claim 5, wherein the first threshold is an omega-3 level at and/or below which the woman's RR EPTB is significantly less than one;
and/or wherein the second threshold is an omega-3 level at and/or above which the woman's RR EPTB is significantly more than one.

7. The method according to claim 4, wherein the first threshold is an omega-3 level of from 3.5% to less than 4.5%.

8. The method according to claim 1, wherein the omega-3 supplementation dose provides from 300 to 1000 mg omega-3 fatty acids per day, from 640 to 960 mg DHA per day and/or from 80 to 120 mg EPA per day.

9. The method according to claim 4, wherein the omega-3 supplementation dose, omega-3 maintenance dose, omega-3 low dose, and/or omega-3 zero dose is adjusted for the one or more additional parameters.

10. A method for determining a personalised omega-3 dose to be taken by a pregnant woman to promote a full-term birth, wherein the method comprises:
(i) determining the woman's omega-3 level; and
(ii) determining the personalised omega-3 dose using the method for determining a personalised omega-3 dose to be taken by a pregnant woman to promote a full-term birth, wherein the method comprises using the woman's omega-3 level to determine the personalised omega-3 dose.

11. A method for providing a personalised omega-3 dose to be taken by a pregnant woman to promote a full-term birth, wherein the method comprises:
(i) determining the woman's omega-3 level;
(ii) determining the personalised omega-3 dose using the method for determining a personalised omega-3 dose to be taken by a pregnant woman to promote a full-term birth, wherein the method comprises using the woman's omega-3 level to determine the personalised omega-3 dose; and
(iii) administering the personalised omega-3 dose to the woman.

12-15. (canceled)
